(19) United States
(12) Patent Application Publication
(10) Pub. No.: US 2014/0127688 A1
(43) Pub. Date: May 8, 2014

(54) METHODS AND SYSTEMS FOR IDENTIFYING CONTAMINATION IN SAMPLES

(71) Applicant: GOOD START GENETICS, INC., Cambridge, MA (US)

(72) Inventors: Mark Umbarger, Brookline, MA (US); Gregory Porreca, Cambridge, MA (US)

(73) Assignee: GOOD START GENETICS, INC., Cambridge, MA (US)

(21) Appl. No.: 14/073,500

(22) Filed: Nov. 6, 2013

Related U.S. Application Data
(60) Provisional application No. 61/723,550, filed on Nov. 7, 2012.

(51) Int. Cl. C12Q 1/68

(52) U.S. Cl. CPC C12Q 1/6876
USPC 435/6.11

(57) ABSTRACT

Methods and systems for determining if a sample has been contaminated with other genetic material, for example, from another sample in a parallel workflow. The methods and systems compare measured allele fractions to predetermined distributions of allele fractions in order to calculate a likelihood that the sample has been contaminated.

### STATEMENT OF RELATED APPLICATIONS

This application claims priority to U.S. Provisional Patent Application No. 61/723,550, filed Nov. 7, 2012, which is incorporated by reference in its entirety.

### FIELD OF THE INVENTION

The invention relates to methods and systems for identifying contamination, e.g., foreign genetic information, in a sample. By comparing distributions of allelic fractions associated with various loci in a sample, it is possible to determine probabilistically whether a sample has been contaminated. The invention is especially useful for quality control in workflows which use massively parallel sequencing.

### BACKGROUND

Genomic sequencing has changed the landscape of clinical diagnosis and treatment due to its speed and extremely low cost-per-base. For example, Illumina's HISEQ™ sequencing platform can simultaneously read hundreds of millions of sequences using competitive, reversible dNTP labeling. However, in order to achieve the low cost, it is often necessary to divide the relatively high fixed per-run cost over multiple different DNA samples that are simultaneously processed. For example, the Illumina workflow requires several time-intensive preparatory steps, thus laboratories typically run as many different genetic samples as possible (simultaneously) to reduce the per-sample cost.

When using parallel sequencing, unique barcodes are typically added to each genetic sample that is to be processed in parallel so that the origin of the sample may be identified when the sequence information is read and reassembled. For example, prior to amplification and sequencing, a genetic sample may be fragmented into manageable read sizes, e.g., 100 bases. A unique (non-naturally occurring) nucleic acid sequence is then ligated to all fragments from each genetic sample, and that unique sequence (barcode) is used to track the origin of the sequences. Other types of sequencing barcodes may involve magnetic beads, for example. The use of barcodes is not limited to Illumina sequencing, however; barcodes are used in a wide variety of genetic techniques such as Life Technologies' SOLiD® sequencing.

While barcodes facilitate tracking genetic samples, they do not eliminate cross-contamination. Sample mix-ups and cross-contamination can occur when the samples are prepared prior to amplification and sequencing, resulting in sequences with the wrong bar codes. Additionally, it is possible for fragmented sequences to be mislabeled during library creation. Such bar code errors can be particularly difficult to deconvolve when a number of similar fragments from different individuals are being assayed for the same information, e.g., breast tumor genotype, as is done in many clinical laboratories.

Sample contamination can have dramatic consequences in clinical sequencing, where the results may be used, for example, to direct treatment for a disease or to guide decisions about the viability of a fetus. For example, a homozygous genotype at a given locus may be indicative of a genetic disease, e.g., sickle-cell anemia. If two samples having different genotypes are cross-contaminated during bar-coding, there is a potential for a false negative diagnosis for the homozygous individual. For example, a first sample, barcoded with barcode 1, could be homozygous recessive (T/T) at the β-globin gene, while a second sample, barcoded with barcode 2, is heterozygous (A/T). If the samples are properly segregated, allelic reads at the β-globin gene labeled with barcode 1 will only indicate T. However, if there has been cross-contamination during library creation, it is possible that some sequences labeled with barcode 1 will indicate A and T, suggesting that sample A has some amount of heterozygosity. Under the right contamination conditions, such an error could result in sample 1 being miscalled as heterozygous, i.e., not positive for the disease. Of course, sickle-cell anemia represents a best-case scenario for cross-contamination in a genetic sample because the disease may be effectively diagnosed using alternative methods, e.g., blood smears under a microscope. Additionally, because the disease is caused by a simple mutation (i.e., a single base change from A to T), contamination would be suspected if the ratio of A to T in a sample was not approximately 50/50, i.e., as expected in a heterozygous sample.

In many diseases, the genetic variations underlying a disease are not as straightforward as a single base mutation. For example, Tay-Sachs disease can be caused by a number of errors in the controlling gene, and the heterozygous genotypes can take a variety of forms. Furthermore, poorly categorized loci and reading errors can complicate the process of distinguishing low-occurrence alleles from contamination from other genetic samples.

Because care-providers are increasingly relying on genetic testing to guide treatment decisions, there is a greater need for improved methods for determining the presence of contaminating genetic information in a genetic sample.

### SUMMARY

The invention provides methods and systems for identifying contamination in a biological sample. Methods of the invention compare expected allelic frequency values observed in samples to values expected to occur (or observed to occur) if there is no contamination in the sample. Expected allelic frequencies at polymorphic loci are compared to actual frequencies observed, for example, from sequencing those loci in material obtained from a biological sample. In the absence of sequencing or amplification errors, the fraction of alleles in a sample would be expected to be 50% for a heterozygote or 100%/0% for a homozygote. Errors introduced in the sequencing and amplification processes are accounted for by observing distributions of allele frequencies in the sample as compared to a reference. The invention provides the ability to obtain genomic sequence reads from a sample and determine whether base calls in those reads are consistent with expected ratios. For example, a genotype call of “AT” at a given locus indicates that the A/T ratio should be 50:50. Statistically-significant deviations from that ratio at the locus are indicative of contamination in the sample.

Methods of the invention are especially useful when applied to polymorphic loci. Those polymorphic loci are likely to be different in different samples. The deviation in a sample from expected allelic frequency (fraction) distributions is indicative of contamination. Assuming that a reference (non-contamination) allelic frequency follows a normal distribution, one simply compares allele frequency distribution at a locus or loci of interest to the reference distribution, using statistical analysis to determine the likelihood of contamination. For example, if a sample is contaminated (e.g., by cross-contamination from another sample) at a nominal rate of 12% and the expected allelic distribution at a given heterozygous locus is 0.48 with a standard deviation of 0.02, the allelic fraction calculated at that locus would be 0.42 as a result of contamination ((1-0.12)*0.48=0.4224). When the observed allelic fraction is converted to a standard (Z) score, the result is −3 ((0.42-0.48)/0.02)). Assuming a standard distribution for the reference, the probability of observing a Z score of −3 in the absence of contamination is less than 0.0015 applying standard statistical analysis. Accordingly, the sample would be identified as being contaminated.

There are numerous ways to apply methods of the invention as disclosed herein, but the basis for those methods is the recognition that there will be a statistically-significant variation of allele fractions across a set of polymorphic loci between observed and expected values if there is contamination in the sample, taking into account sequencing- and amplification-induced errors.

The disclosed methods and systems are also useful to detect and quantify fetal DNA fractions in maternal blood as well as maternal contamination of fetal genetic material from amniocentesis or chorionic villus sampling (CVS). The methods and systems are useful to identify aneuploidy in a sample and to distinguish genetic mutations from contamination.

Typically, the invention involves comparing allelic fractions at polymorphic loci in a sample to predetermined allelic fractions for the same loci. In one embodiment, the predetermined distribution of alleles results from analysis of a set of genetic data that is known to be free from contamination. Often the allele of interest will be a minor (non-reference) allele at a locus known to have a good deal of variation among the population. Using minor alleles with high population frequencies increases the likelihood that a random sample contaminating the intended sample will have a different identity at the locus. For each locus a score can be produced, and a summary statistic can be prepared from the collected scores to allow a user to quickly and reliably identify samples that are likely contaminated.

In one instance, the invention includes a method for determining contamination in a genetic sample (i.e., a sample containing genetic or genomic material). Those methods comprise determining a sequence of one or more nucleic acids in the sample at one or more polymorphic loci; and comparing a set of observed allele frequencies at the polymorphic loci in the sequence to reference distributions of alleles at the polymorphic loci. A statistically significant difference between the observed values and the reference distributions is indicative of contamination in the sample. Methods of the invention are useful with any sequencing or genotyping technique, especially massive parallel sequencing, i.e., next generation sequencing.

Methods of the invention score differences between measured allelic fractions and predetermined allelic fraction distributions and accumulate the scores for easy evaluation. For example, a z-score can be assigned to each locus in the sample, and a summary statistic of the z-scores can be calculated for comparison to a predetermined or reference distribution. The summary statistic can then be compared to a predetermined distribution of summary statistics based upon z-scores for the individual sequences in the genetic data known to be free from contamination.

Methods of the invention are useful to analyze a sample based upon identified genotypes at polymorphic loci in the sample. The genotype may be heterozygous or homozygous, and may be determined with respect to a reference allele (e.g., a known allele of clinical interest, or an allele identified in a published sequence) or a non-reference allele (e.g., an allele that is not of clinical interest). In some embodiments, methods of the invention are used only with non-reference alleles.

Methods of the invention encompass a variety of known assay techniques. In one instance, the invention is a method of identifying a genetic abnormality, comprising providing a sample, determining a sequence from the sample, identifying the allele fractions at polymorphic loci in the sequence, comparing a portion of the sequence to a predetermined sequence, and, comparing the observed allele fractions at the polymorphic loci in the sequence to predetermined distributions of alleles at the same loci. In this instance, a difference between the portion of the sequence and the predetermined sequence in the absence of a statistically significant difference between the distribution and the predetermined distribution is indicative of a genetic abnormality.

In another instance, the invention is a system for determining contamination in a genetic sample. The system includes a processor and a computer-readable storage medium. The computer-readable storage medium contains instructions which, when executed by the processor, cause the system to compare a set of observed allele frequencies polymorphic loci in a sample to a predetermined distribution of alleles at the same polymorphic loci and compute a likelihood (e.g., probability) that a difference between the distribution and the predetermined distribution is indicative of contamination in the sample. The system may provide a sophisticated analysis of the probability of contamination being present by incorporating additional instructions that instruct the processor to carry out the analyses outlined above. For example, the readable medium may contain instructions that cause the processor to prepare an accumulated comparison for a plurality of loci in a new sample. In some embodiments, a z-score will be assigned to each locus in a sample and a summary statistic of the z-scores will be calculated for comparison to the predetermined (or theoretically expected) distribution. A system of the invention may stand alone, or it may be integrated into a genetic analysis platform, e.g., a next-generation sequencing platform.

In another instance, the invention is an alternative method for determining contamination in a genetic sample. This method includes sequencing a plurality of genetic sequences corresponding to a sample, identifying a plurality of possible genotypes at a locus common to the plurality of genetic sequences, calculating the probabilities of each genotype at this locus, ranking the possible genotypes based upon their probabilities (thereby establishing a most probable genotype, a second most frequent genotype, etc.) and comparing the second most probable genotype to the most probable genotype to determine if the genetic sample has been contaminated. When using this method, a small difference in probability between the second most probable genotype and most probable genotype is indicative of contamination in the sample. For example if the second most probable genotype is nearly equally probable to the most probable genotype, it may be indicative of contamination. This method may also be implemented as an independent system, e.g., including a processor and a computer-readable storage medium, wherein the medium contains instructions for the processor to execute the method for determining contamination in a genetic sample.

Methods of the invention are useful to quantify sample contamination by building a standard curve of contamination events and comparing sample contamination against the curve. Methods of the invention are also useful to determine mitochondrial heteroplasmy. For example, methods of the invention applied to mitochondrial nucleic acids are useful to detect the presence of mixed genomic material (mutations) in a patient sample.

Thus, the methods and systems of the invention will assist users, e.g., clinicians, in identifying contamination in genetic samples. The methods and systems will help to reduce rates of false diagnosis, especially in the fields of cancer genotyping and prenatal genetics.

### BRIEF DESCRIPTION OF THE DRAWINGS

FIG. 1 is a flowchart showing a method for determining if a genetic sample has been contaminated.

FIG. 2 compares a distribution of mean z-scores from a set of sequences known to be free from contamination to the mean z-score for a sample known to have been contaminated.

### DETAILED DESCRIPTION

The invention provides improved methods and systems for determining contamination in a biological sample. In particular, by measuring allelic fractions at a number of genomic positions and scoring the allelic fractions against those expected in an uncontaminated samples, it is possible to efficiently identify samples that have been contaminated. The methods and systems will be especially useful for clinicians and laboratories that use barcoding to track genetic samples in order to simultaneously process large numbers of similar genetic samples.

It is appreciated by those of skill in the art that there exist a number of polymorphic loci (positions) in a genome, e.g., the human genome. That is, some portions of the genome are more likely to have variations between individuals, while others are more likely to be the same (i.e., “conserved” regions). Typically, the most common allele at a locus (by population) is called the major allele and the lesser common alleles are known as minor alleles. The greater the degree of polymorphicity, the greater the chance two random genetic samples from different individuals will have different sequences at the polymorphic locus.

Additionally, the greater the heterogeneity, the greater the chance that two random genetic samples from different individuals will have different genotypes at the polymorphic locus. In a diploid organism (e.g., mammals), polymorphic alleles result in greater diversity in genotypes, because each organism has at least two alleles at the polymorphic locus.

When there is only one minor allele A, and the majority allele is B, the probability that two random samples have different genotypes can be calculated based upon the minor allele frequency (maf):

1−P[same]=1−(P_{AA}²+2P_{AB}+P_{BB}²)

where P_{AA}=maf², P_{AB}=P_{BA}=maf(1−maf), and P_{BB}=(1−maf)². In the limit where the minor allele approaches parity with the major allele, the likelihood that two random samples will have different genotypes will approach 75%. In the case that a locus has multiple minor alleles and the major allele represents less than 50% of the alleles, the likelihood that two random samples will have different genotypes increases greater still.

Therefore, if the goal is to make it easier to identify random contamination, one should study loci with high polymorphism because a sequence from a random sample is more likely to be different from the sample. Furthermore, measuring genotypes at the polymorphic loci will further increase the likelihood that a random sample is different. Accordingly, because most genetic samples have more than one locus, it should be possible to further increase the likelihood of detecting random genetic contamination by studying a plurality of loci for each sample.

As is known in the art, when measuring genotypes at a locus of a diploid organism, the ratio between minor alleles, or between a minor and a major allele, should theoretically be 2:0, 1:1, or 0:2, corresponding to homozygous (AA), heterozygous (AB), or homozygous (BB). Normalizing those ratios, as is done with genotype calling, a particular allele should have a fraction of 0, ½, or 1. In reality, sample bias and random error combine to produce a distribution of allele fractions for each genotype at a given locus. For example, a collection of 100 different samples may be genotyped for the same locus, whereupon it is discovered that allelic fractions for allele A are 0.97±0.02, 0.48±0.02, and 0.02±0.03. This allele fraction distribution, determined by examining a set of clean samples, is termed the “null” distribution, i.e., the expected distribution as the probability of contamination approaches zero.

The null distribution for a given allele will vary somewhat based upon the workflow because of sampling biases that are unique to particular protocols and machines. It will be necessary to determine a null distribution for each combination of preparatory steps (e.g., DNA fragmentation technique) and sequencing technique (e.g., specific sequencing platform). Typically, a null distribution will be assembled from at least 10, e.g., at least 20, e.g., at least 30, e.g., at least 40, e.g., at least 50, e.g., at least 60, e.g., at least 70, e.g., at least 80, e.g., at least 90, e.g., at least 100 genetic samples known to be free from contamination. Typically each sequence in the null distribution will have at least 2 different polymorphic loci, e.g., at least 3 different polymorphic loci, e.g., at least 5 different polymorphic loci, e.g., at least 10 different polymorphic loci. In many cases, it will be beneficial to include a variety of genotypes at the polymorphic loci, so that it is possible to determine an allelic fraction for each genotype at each identified polymorphic locus.

When genetic contamination is introduced in a sample during a workflow, the allelic fraction for the sample will likely not match with any of the three genotype distributions determined from the null set. That is, the contamination will result in an unexpected ratio of a specific allele to all alleles (i.e., the allele fraction) as compared to the expected distribution for the workflow. For example, if the sample discussed above was contaminated with about 12% of a foreign minor allele, C, the measured heterozygous allele fraction for allele A would report at about (1-0.42)*0.48.

The variance in allelic fraction due to contamination may take one of two forms. In some samples, where the contamination was introduced early in the work flow, the allelic fraction of A varies from the predetermined allelic fraction for the called genotype throughout the entire sequencing process. In other samples, where the contamination was introduced later in the workflow, the allelic fraction will change only after the introduction of the contaminant, implying that if one were to measure the allele fraction at different stages of the workflow, one could potentially identify when the contamination occurred. For example, if the sample discussed above was contaminated early in the workflow, the measured heterozygous allele fraction for allele A would report at about 0.42 throughout the process, indicating that something went awry early in the workflow. Alternatively, if the contamination was introduced later in the workflow, the initial measured allelic fraction would initially report at 0.48, but with successive reads, the allele fraction will decrease. In the case where the allele fraction changes with time, it may be possible to calculate the correct allelic fraction, or rely on the earlier measurements (discussed below).

The above examples are illustrative, however, and in practice the discrepancies between measured allelic fractions and expected allelic fractions are not as obvious. Accordingly, the methods of the invention use probabilistic scoring to determine the likelihood that a measured allelic fraction is within the expected range. Continuing with the above example (i.e., assuming that the allelic fraction for A for a heterozygous read was determined to be 0.42), the difference between the measured fraction and the “normal” or “null” distribution would be −0.06, i.e., 0.42-0.48. A z-score can be assigned to this variation, using the previously determined error on the null distribution:

$$z = \frac{x - μ}{σ}$$

where x is the measured value (0.42), μ is the mean value of the predetermined distribution (0.48) and σ is the standard deviation of the predetermined distribution (0.02). Thus, for the example above, the z-score would be −3. The measured variance can be compared to the standard deviation, and used to determine a p-value for the measured distribution. In this case, the p-value would be 0.0015. Because the p-value is so much smaller than the standard deviation, the null hypothesis (i.e., that there was no contamination in sample) would be rejected. In other words, because the p-value is so small, it is likely that the sample was contaminated.

One of skill in the art will appreciate that the number of different alleles for a given locus is limited, thus there is a finite possibility that contamination from another sample will have the same allele (or genotype) at a specific locus. Accordingly, there is a quantifiable risk that a contaminating genetic sample will have the same allele at a given locus or even the same genotype. If only one locus is analyzed, contamination will be missed when the contamination has the same genotype as the desired sample.

To avoid missing signs of contamination, the methods and systems of the invention compare a plurality of polymorphic loci in each sample. After comparison information is collected for the loci, a summary statistic can be prepared and reported to allow a user to quickly evaluate the likelihood of contamination. In one embodiment, the summary statistic is a mean of the z-scores for the allelic fractions measured for the genotype at n polymorphic loci. For example, the z-scores for each of four polymorphic loci in a sample may be averaged to (z₁+z₂+z₃+z₄)/4. The average z score can then be used to calculate the probability that the sample was not contaminated by comparing the average z score to an average z score for the same loci from the null set, i.e., the set of samples that are known to have been free of contamination. Alternatively, the average z-score for the null set can be quickly calculated assuming that a database of allelic fraction distributions has been previously prepared referenced by genotype and locus. The summary statistic need not be limited to the mean, however, a median z-score could be evaluated if there are a sufficient number of polymorphic loci in the sample. Alternatively, a z-score threshold could be set so that any individual z-score above a preset number would result in the sample being flagged for possible contamination. Combinations of these summary statistics are also possible.

In another embodiment, the average measured z-score for the sample can be evaluated as a function of the number of measurements (where measurements occur at different times in the sample prep workflow), or a number of individual z-scores can be simultaneously evaluated as a function of the number of measurements to probe whether the z-scores are stable throughout the sample prep workflow. If one or more z-scores, or the average z-score, is changing with the number of measurements, it is likely that the sample has been contaminated somewhere between the points in time where the z-scores changed. In this instance, it may be possible to “back-out” the correct information, however, because the point at which the contamination occurred should be evident as the point where the z-score began to change. Additionally, in the instances where noise, or some other interference makes it difficult to determine when the contamination began, it is possible to model the z-score change based on secondary measurements in which contamination is added to a known sequence at a known rate.

In alternative embodiments, contamination of a genetic sample may be assessed by comparing the genotype rankings of the sequence data as it produced by sequencing software accompanying the sequencing platform. Specifically, when there is moderate contamination of a sample at a polymorphic locus, genotype calling software should propose one or more outlier genotypes that are less likely than the most probable genotype, but substantially more probable than the other possible genotypes, which should only have genotype hits because of sampling errors. For example, in the instance that a sample, heterozygous AB, is contaminated by genetic material having a different allele C at the locus, the probable genotypes would include the correct genotype AB as the most probable genotype, second and third most probable genotypes, AC and BD (due to contamination), and other less probable genotypes, such as AA, BB, CC, etc. In the event that the second and third most probable genotypes are substantially more likely than the remaining, less common genotypes, it is likely that the sample has been contaminated with genetic material having a different allele. Obviously, this method will not work when the contaminating sample has the same genotype at the locus. This method may be used independently from the methods described above, or it can be used to complement the methods described above.

In practice, the described methods will typically be incorporated into a system, e.g., a sequencing platform, or software for analyzing sequence data. In an embodiment, the system comprises a processor and a computer-readable storage medium. The system and computer-readable medium may reside in the same computer, e.g., a desktop computer or server, or the processor and the computer-readable storage medium may reside in different locations and communicate via a network, e.g., the internet. In some instances, a system will employ a plurality of processors or a plurality of computer-readable storage media. The plurality of processors or the plurality of computer-readable storage media may be distributed to different geographic locations, or that the plurality of processors or the plurality of computer-readable storage media may be at the same geographic location.

In systems of the invention, stored instructions are executed to cause the processor to compare a measured distribution of alleles in a genetic sample to a predetermined distribution of alleles and compute a likelihood (e.g., probability) that a difference between the measured distribution and the predetermined distribution is indicative of contamination in the genetic sample. This allows the system to determine whether the genetic sample being analyzed is likely to have been contaminated by another sample. Using such a system, it is easy for a user, e.g., a laboratory technician, to flag samples that need to be discarded or re-run.

In other embodiments, the system may include additional functionality or automation of the methods described above. For example, the stored instructions may further instruct the processor to compute a rate of change in the difference between the measured distribution and the predetermined distribution as a function of a number of sequence iterations. The stored instructions may also instruct the processor to receive information about one or more loci of interest, and then to identify those loci in the sample. The instructions may instruct the processor to identify a genotype (e.g., homozygous or heterozygous) at the locus, and determine an allelic fraction for an allele associated with the genotype.

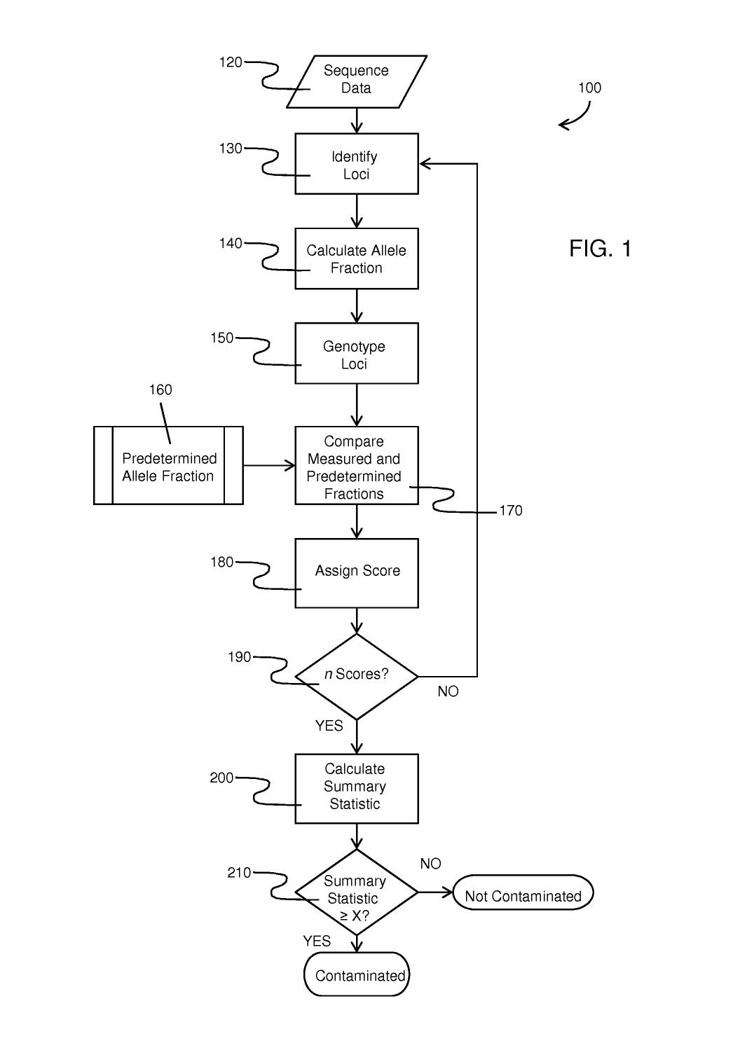

An exemplary flowchart, showing a system for determining contamination in a genetic sample 100 is shown in FIG. 1. Initially, sequence data 120 is input into the system. The sequence data 120 can take the form of a data file, e.g., an output file from a sequencing platform, or some other listing of sequence information. For better results, sequence data 120 should include multiple reads of the same sequence or portions of the same sequence, and the sequence should include at least a few polymorphic loci. In one embodiment, the sequence data 120 is from a parallel sequencing platform, e.g., Illumina sequencing. The system takes the input sequence data 120 and identifies relevant polymorphic loci at step 130. Relevant loci are polymorphic, meaning that they are likely to have a distribution of alleles, and the relevant loci are identifiable in the sequence data 120 that is provided. In some embodiments, a user directs the loci to be identified based upon knowledge of the sequences that have been processed or the way in which the sample was originally fragmented or amplified.

Once the relevant loci have been identified at step 130, sequences corresponding to different alleles that have been read at the loci are tabulated and an allelic fraction is calculated at step 140. Based upon the allelic fraction(s) (and potentially base qualities), a genotype is assigned 150 to each locus for comparison to the null distribution. At step 170, the system 100 compares the measured allelic fraction 140 to a predetermined allelic fraction 160 for the identified genotype 150. The predetermined allelic fraction 160 will typically correspond to a mean allelic fraction, with an associated standard deviation, originating in a null set, i.e., a set of sequences that are known to be free from contamination during sequencing. Additionally, the predetermined allele fraction will typically be prepared using the same workflow as the workflow used to collect sequence data 120 (described above). In one embodiment, the predetermined allelic fractions 160 are indexed in a database by locus and genotype. In another embodiment, the null set is simply a set of sequences, or a set of alleles, and the system determines the distribution of null set alleles as needed for comparison.

After comparing the measured and the predetermined allelic fractions at 170, a system 100 of the invention assigns a score to the measured allelic fraction at 180. The score may be a z-score, as described above, or the score may be a t-score, or a percentile, or expressed in a number of standard deviations from the mean. At step 190, the system determines if enough loci have been assessed to produce a meaningful determination of the presence of contamination. In some embodiments, the number of loci sampled, n, will be a user input. However, in more sophisticated systems, the system 100 may be programmed to continue identifying loci and comparing measured and predetermined distribution until the process converges, i.e., as shown with the arrow from 190 to 130. One skilled in the art will appreciate that scoring loci need not happen serially, as is shown in FIG. 1. Rather, n loci may be simultaneously evaluated and scored.

At step 200, a summary statistic is calculated based upon the accumulated z-scores for the n loci. As discussed above, the summary statistic may take any of a number of forms including the mean, median, or max. At step 210 the summary statistic is compared to a predetermined value, X, to determine the likelihood that a sample was contaminated. The value X may be a user adjustable input, or the value of X may be preset for the system. For example, if the summary statistic is the mean or median z-score, X may be set to ≧2, or ≧3, or ≧4. If the summary statistic is the maximum z-score, X may be set higher, i.e., ≧3, ≧4, or ≧5. If a different summary statistic is used, X can be adjusted appropriately. In other embodiments, X may be a distribution of scores for the elements of the null set that was originally used to determine the allelic distributions. In other embodiments, a p-value may be calculated reflecting a probability that the null hypothesis is correct (i.e., that no contamination is present).

FIG. 1 should be viewed as exemplary of a system of the invention. Variations on the system described in FIG. 1 will be evident to one of skill in the art. Additionally, FIG. 1 should not be viewed as limiting a system of the invention. For example, it may be unnecessary to calculate a summary statistic because the system is programmed to flag a sample as contaminated as soon as any locus achieves a score beyond a preset value. Alternatively, more elaborate flow charts can be prepared in which each sample from the null set is analyzed against the population of null samples using steps 130-180, as is done in Example 1 (below).

### Genetic Testing

Genetic testing, including DNA-based tests, involves techniques used to test for genetic disorders through the direct examination of nucleic acids. Other genetic tests include biochemical tests for such gene products as enzymes and other proteins and for microscopic examination of stained or fluorescent chromosomes.

Genetic tests may be used in a variety of circumstances or for a variety of purposes. For example, genetic testing includes carrier screening to identify unaffected individuals who carry one copy of a gene for a disease with a homozygous recessive genotype. Genetic testing can be used to identify individuals with an extra chromosome (aneuploidy). Genetic testing can further include pre-implantation genetic diagnosis, prenatal diagnosis, newborn screening, genealogical testing, screening and risk-assessment for adult-onset disorders such as Huntington's, cancer or Alzheimer's disease, as well as forensic and identity testing. Testing is sometimes used just after birth to identify genetic disorders that can be treated early in life. Newborn tests include tests for phenylketonuria and congenital hypothyroidism. Genetic tests can be used to diagnose genetic or chromosomal conditions at any point in a person's life, to rule out or confirm a diagnosis. Carrier testing is used to identify people who carry one copy of a gene mutation that, when present in two copies, causes a genetic disorder. Prenatal testing is used to detect changes in a fetus's genes or chromosomes before birth. Predictive testing is used to detect gene mutations associated with disorders that appear later in life. For example, testing for a mutation in BRCA1 can help identify people at risk for breast cancer. Pre-symptomatic testing can help identify those at risk for hemochromatosis. Genetic testing further plays important roles in research. Researchers use existing lab techniques, as well as develop new ones, to study known genes, discover new genes, and understand genetic conditions.

Because genetic testing is relied upon, to a great extent, for clinical and pre-clinical diagnosis, the consequences of errors due to contamination are dire. For example, a cancer patient may be put on the wrong chemotherapeutic regiment because of an error in genotyping a cancer biopsy. Alternatively, a mother may wrongly decide to terminate a pregnancy because of incorrect genetic information obtained via an amniocentesis, or other prenatal test.

As discussed above, contamination in a genetic sample may originate in other samples that are processed along with the sample of interest. However contamination may also be introduced because of fetal DNA fractions in maternal blood, maternal contamination of amniocentesis, or maternal contamination of chorionic villus sampling (CVS).

At present, there are more than 1,000 different genetic tests available. Genetic tests can be performed using a biological sample such as blood, hair, skin, amniotic fluid, cheek swabs from a buccal smear, or other biological materials. Blood samples can be collected via syringe or through a finger-prick or heel-prick. Such biological samples are typically processed and sent to a laboratory. A number of genetic tests can be performed, including karyotyping, restriction fragment length polymorphism (RFLP) tests, biochemical tests, mass spectrometry tests such as tandem mass spectrometry (MS/MS), tests for epigenetic phenomenon such as patterns of nucleic acid methylation, and nucleic acid hybridization tests such as fluorescent in-situ hybridization. In certain embodiments, a nucleic acid is isolated and sequenced.

Nucleic acid template molecules (e.g., DNA or RNA) can be isolated from a sample containing other components, such as proteins, lipids and non-template nucleic acids. Nucleic acid can be obtained directly from a patient or from a sample such as blood, urine, cerebrospinal fluid, seminal fluid, saliva, sputum, stool and tissue. Any tissue or body fluid specimen may be used as a source for nucleic acid. Nucleic acid can also be isolated from cultured cells, such as a primary cell culture or a cell line. Generally, nucleic acid can be extracted, isolated, amplified, or analyzed by a variety of techniques such as those described by Green and Sambrook, Molecular Cloning: A Laboratory Manual (Fourth Edition), Cold Spring Harbor Laboratory Press, Woodbury, N.Y. 2,028 pages (2012); or as described in U.S. Pat. No. 7,957,913; U.S. Pat. No. 7,776,616; U.S. Pat. No. 5,234,809; U.S. Pub. 2010/0285578; and U.S. Pub. 2002/0190663.

Nucleic acid obtained from biological samples may be fragmented to produce suitable fragments for analysis. Template nucleic acids may be fragmented or sheared to desired length, using a variety of mechanical, chemical and/or enzymatic methods. Nucleic acid may be sheared by sonication, brief exposure to a DNase/RNase, hydroshear instrument, one or more restriction enzymes, transposase or nicking enzyme, exposure to heat plus magnesium, or by shearing. RNA may be converted to cDNA, e.g., before or after fragmentation. In one embodiment, nucleic acid from a biological sample is fragmented by sonication. Generally, individual nucleic acid template molecules can be from about 2 kb bases to about 40 kb, e.g., 6 kb-10 kb fragments.

A biological sample as described above may be lysed, homogenized, or fractionated in the presence of a detergent or surfactant. The concentration of the detergent in the buffer may be about 0.05% to about 10.0%, e.g., 0.1% to about 2%. The detergent, particularly a mild one that is non-denaturing, can act to solubilize the sample. Detergents may be ionic (e.g., deoxycholate, sodium dodecyl sulfate (SDS), N-lauroylsarcosine, and cetyltrimethylammonium bromide) or nonionic (e.g., octyl glucoside, polyoxyethylene(9)dodecyl ether, digitonin, polysorbate 80 such as that sold under the trademark TWEEN by Uniqema Americas (Paterson, N.J.), (C₁₄H₂₂O(C₂H₄)ₙ) sold under the trademark TRITON X-100 by Dow Chemical Company (Midland, Mich.), polidocanol, n-dodecyl beta-D-maltoside (DDM), or NP-40 nonylphenyl polyethylene glycol). A zwitterionic reagent may also be used in the purification schemes, such as zwitterion 3-14 and 3-[(3-cholamidopropyl) dimethyl-ammonio]-1-propanesulfonate (CHAPS). Urea may also be added. Lysis or homogenization solutions may further contain other agents, such as reducing agents. Examples of such reducing agents include dithiothreitol (DTT), β-mercaptoethanol, dithioerythritol (DTE), glutathione (GSH), cysteine, cysteamine, tricarboxyethyl phosphine (TCEP), or salts of sulfurous acid.

In various embodiments, the nucleic acid is amplified, for example, from the sample or after isolation from the sample. Amplification refers to production of additional copies of a nucleic acid sequence and is generally carried out using polymerase chain reaction (PCR) or other technologies known in the art. The amplification reaction may be any amplification reaction known in the art that amplifies nucleic acid molecules, such as PCR, nested PCR, PCR-single strand conformation polymorphism, ligase chain reaction (Barany, F., The Ligase Chain Reaction in a PCR World, Genome Research, 1:5-16 (1991); Barany, F., Genetic disease detection and DNA amplification using cloned thermostable ligase, PNAS, 88:189-193 (1991); U.S. Pat. No. 5,869,252; and U.S. Pat. No. 6,100,099), strand displacement amplification and restriction fragments length polymorphism, transcription based amplification system, rolling circle amplification, and hyper-branched rolling circle amplification. Further examples of amplification techniques that can be used include, but are not limited to, quantitative PCR, quantitative fluorescent PCR (QF-PCR), multiplex fluorescent PCR (MF-PCR), real time PCR (RTPCR), restriction fragment length polymorphism PCR (PCR-RFLP), in situ rolling circle amplification (RCA), bridge PCR, picotiter PCR, emulsion PCR, transcription amplification, self-sustained sequence replication, consensus sequence primed PCR, arbitrarily primed PCR, degenerate oligonucleotide-primed PCR, and nucleic acid based sequence amplification (NABSA). Amplification methods that can be used include those described in U.S. Pat. Nos. 5,242,794; 5,494,810; 4,988,617; and 6,582,938. In certain embodiments, the amplification reaction is PCR as described, for example, in Dieffenbach and Dveksler, PCR Primer, a Laboratory Manual, 2nd Ed, 2003, Cold Spring Harbor Press, Plainview, N.Y.; U.S. Pat. No. 4,683,195; and U.S. Pat. No. 4,683,202, hereby incorporated by reference. Primers for PCR, sequencing, and other methods can be prepared by cloning, direct chemical synthesis, and other methods known in the art. Primers can also be obtained from commercial sources such as Eurofins MWG Operon (Huntsville, Ala.) or Life Technologies (Carlsbad, Calif.).

With these methods, a single copy of a specific target nucleic acid may be amplified to a level that can be detected by several different methodologies (e.g., sequencing, staining, hybridization with a labeled probe, incorporation of biotinylated primers followed by avidin-enzyme conjugate detection, or incorporation of 32P-labeled dNTPs). Further, the amplified segments created by an amplification process such as PCR are, themselves, efficient templates for subsequent PCR amplifications. After any processing steps (e.g., obtaining, isolating, fragmenting, or amplification), nucleic acid can be sequenced.

Sequencing may be by any of a variety of methods. DNA sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing, Illumina/Solexa sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLiD sequencing. Separated molecules may be sequenced by sequential or single extension reactions using polymerases or ligases as well as by single or sequential differential hybridizations with libraries of probes.

A sequencing technique that can be used includes, for example, use of sequencing-by-synthesis systems sold under the trademarks GS JUNIOR, GS FLX+ and 454 SEQUENCING by 454 Life Sciences, a Roche company (Branford, Conn.), and described by Margulies, M. et al., Genome sequencing in micro-fabricated high-density picotiter reactors, Nature, 437:376-380 (2005); U.S. Pat. No. 5,583,024; U.S. Pat. No. 5,674,713; and U.S. Pat. No. 5,700,673, the contents of which are incorporated by reference herein in their entirety. 454 sequencing involves two steps. In the first step of those systems, DNA is sheared into fragments of approximately 300-800 base pairs, and the fragments are blunt ended. Oligonucleotide adaptors are then ligated to the ends of the fragments. The adaptors serve as primers for amplification and sequencing of the fragments. The fragments can be attached to DNA capture beads, e.g., streptavidin-coated beads using, e.g., Adaptor B, which contains 5′-biotin tag. The fragments attached to the beads are PCR amplified within droplets of an oil-water emulsion. The result is multiple copies of clonally amplified DNA fragments on each bead. In the second step, the beads are captured in wells (pico-liter sized). Pyrosequencing is performed on each DNA fragment in parallel. Addition of one or more nucleotides generates a light signal that is recorded by a CCD camera in a sequencing instrument. The signal strength is proportional to the number of nucleotides incorporated. Pyrosequencing makes use of pyrophosphate (PPi) which is released upon nucleotide addition. PPi is converted to ATP by ATP sulfurylase in the presence of adenosine 5′ phosphosulfate. Luciferase uses ATP to convert luciferin to oxyluciferin, and this reaction generates light that is detected and analyzed.

Another example of a DNA sequencing technique that can be used is SOLiD technology by Applied Biosystems from Life Technologies Corporation (Carlsbad, Calif.). In SOLiD sequencing, genomic DNA is sheared into fragments, and adaptors are attached to the 5′ and 3′ ends of the fragments to generate a fragment library. Alternatively, internal adaptors can be introduced by ligating adaptors to the 5′ and 3′ ends of the fragments, circularizing the fragments, digesting the circularized fragment to generate an internal adaptor, and attaching adaptors to the 5′ and 3′ ends of the resulting fragments to generate a mate-paired library. Next, clonal bead populations are prepared in microreactors containing beads, primers, template, and PCR components. Following PCR, the templates are denatured and beads are enriched to separate the beads with extended templates. Templates on the selected beads are subjected to a 3′ modification that permits bonding to a glass slide. The sequence can be determined by sequential hybridization and ligation of partially random oligonucleotides with a central determined base (or pair of bases) that is identified by a specific fluorophore. After a color is recorded, the ligated oligonucleotide is removed and the process is then repeated.

Another example of a DNA sequencing technique that can be used is ion semiconductor sequencing using, for example, a system sold under the trademark ION TORRENT by Ion Torrent by Life Technologies (South San Francisco, Calif.). Ion semiconductor sequencing is described, for example, in Rothberg, et al., An integrated semiconductor device enabling non-optical genome sequencing, Nature 475:348-352 (2011); U.S. Pubs. 2009/0026082, 2009/0127589, 2010/0035252, 2010/0137143, 2010/0188073, 2010/0197507, 2010/0282617, 2010/0300559, 2010/0300895, 2010/0301398, and 2010/0304982, the content of each of which is incorporated by reference herein in its entirety. In ion semiconductor sequencing, DNA is sheared into fragments of approximately 300-800 base pairs, and the fragments are blunt ended. Oligonucleotide adaptors are then ligated to the ends of the fragments. The adaptors serve as primers for amplification and sequencing of the fragments. The fragments can be attached to a surface and are attached at a resolution such that the fragments are individually resolvable. Addition of one or more nucleotides releases a proton (H⁺), which signal is detected and recorded in a sequencing instrument. The signal strength is proportional to the number of nucleotides incorporated.

Another example of a sequencing technology that can be used is Illumina sequencing. Illumina sequencing is based on the amplification of DNA on a solid surface using fold-back PCR and anchored primers. Genomic DNA is fragmented, and adapters are added to the 5′ and 3′ ends of the fragments. DNA fragments that are attached to the surface of flow cell channels are extended and bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labeled, reversibly terminating nucleotides are used to perform sequential sequencing. After nucleotide incorporation, a laser is used to excite the fluorophores, and an image is captured and the identity of the first base is recorded. The 3′ terminators and fluorophores from each incorporated base are removed and the incorporation, detection and identification steps are repeated. Sequencing according to this technology is described in U.S. Pub. 2011/0009278, U.S. Pub. 2007/0114362, U.S. Pub. 2006/0024681, U.S. Pub. 2006/0292611, U.S. Pat. No. 7,960,120, U.S. Pat. No. 7,835,871, U.S. Pat. No. 7,232,656, U.S. Pat. No. 7,598,035, U.S. Pat. No. 6,306,597, U.S. Pat. No. 6,210,891, U.S. Pat. No. 6,828,100, U.S. Pat. No. 6,833,246, and U.S. Pat. No. 6,911,345, each of which are herein incorporated by reference in their entirety.

Another example of a sequencing technology that can be used includes the single molecule, real-time (SMRT) technology of Pacific Biosciences (Menlo Park, Calif.). In SMRT, each of the four DNA bases is attached to one of four different fluorescent dyes. These dyes are phospholinked. A single DNA polymerase is immobilized with a single molecule of template single stranded DNA at the bottom of a zero-mode waveguide (ZMW). A ZMW is a confinement structure which enables observation of incorporation of a single nucleotide by DNA polymerase against the background of fluorescent nucleotides that rapidly diffuse in and out of the ZMW (in microseconds). It takes several milliseconds to incorporate a nucleotide into a growing strand. During this time, the fluorescent label is excited and produces a fluorescent signal, and the fluorescent tag is cleaved off. Detection of the corresponding fluorescence of the dye indicates which base was incorporated. The process is repeated.

Another example of a sequencing technique that can be used is nanopore sequencing (Soni, G. V., and Meller, A., Clin. Chem. 53: 1996-2001 (2007)). A nanopore is a small hole, of the order of 1 nanometer in diameter. Immersion of a nanopore in a conducting fluid and application of a potential across it results in a slight electrical current due to conduction of ions through the nanopore. The amount of current which flows is sensitive to the size of the nanopore. As a DNA molecule passes through a nanopore, each nucleotide on the DNA molecule obstructs the nanopore to a different degree. Thus, the change in the current passing through the nanopore as the DNA molecule passes through the nanopore represents a reading of the DNA sequence.

Another example of a sequencing technique that can be used involves using a chemical-sensitive field effect transistor (chemFET) array to sequence DNA (for example, as described in U.S. Pub. 2009/0026082). In one example of the technique, DNA molecules can be placed into reaction chambers, and the template molecules can be hybridized to a sequencing primer bound to a polymerase. Incorporation of one or more triphosphates into a new nucleic acid strand at the 3′ end of the sequencing primer can be detected by a change in current by a chemFET. An array can have multiple chemFET sensors. In another example, single nucleic acids can be attached to beads, and the nucleic acids can be amplified on the bead, and the individual beads can be transferred to individual reaction chambers on a chemFET array, with each chamber having a chemFET sensor, and the nucleic acids can be sequenced.

Another example of a sequencing technique that can be used involves using an electron microscope as described, for example, by Moudrianakis, E. N. and Beer M., in Base sequence determination in nucleic acids with the electron microscope, III. Chemistry and microscopy of guanine-labeled DNA, PNAS 53:564-71 (1965). In one example of the technique, individual DNA molecules are labeled using metallic labels that are distinguishable using an electron microscope. These molecules are then stretched on a flat surface and imaged using an electron microscope to measure sequences.

Sequencing generates a plurality of reads. Reads generally include sequences of nucleotide data less than about 150 bases in length, or less than about 90 bases in length. In certain embodiments, reads are between about 80 and about 90 bases, e.g., about 85 bases in length. In some embodiments, these are very short reads, i.e., less than about 50 or about 30 bases in length. After obtaining sequence reads, they can be assembled into sequence assemblies. Sequence assembly can be done by methods known in the art including reference-based assemblies, de novo assemblies, assembly by alignment, or combination methods. Assembly can include methods described in U.S. Pat. No. 8,209,130 titled Sequence Assembly, and co-pending U.S. patent application Ser. No. 13/494,616, both by Porecca and Kennedy, the contents of each of which are hereby incorporated by reference in their entirety for all purposes. In some embodiments, sequence assembly uses the low coverage sequence assembly software (LOCAS) tool described by Klein, et al., in LOCAS-A low coverage sequence assembly tool for re-sequencing projects, PLoS One 6(8) article 23455 (2011), the contents of which are hereby incorporated by reference in their entirety. Sequence assembly is described in U.S. Pat. No. 8,165,821; U.S. Pat. No. 7,809,509; U.S. Pat. No. 6,223,128; U.S. Pub. 2011/0257889; and U.S. Pub. 2009/0318310, the contents of each of which are hereby incorporated by reference in their entirety.

Nucleic acid sequence data may be analyzed with a variety of methods to determine the presence of biomarkers, where reads should start and stop, and how different sequences from the original sample fit together. Multiplex ligation-dependent probe amplification (MLPA) uses a pair of primer probe oligos, in which each oligo of the pair has a hybridization portion and a fluorescently-labeled primer portion. When the two oligos hybridize adjacent to each other on the target sequence, they are ligated by a ligase. The primer portions are then used to amplify the ligated probes. Resulting product is separated by electrophoresis, and the presence of fluorescent label at positions indicting the presence of target in the sample is detected. Using a single set of primers and hybridization portions for multiple targets, the analysis can be multiplexed. Such techniques can be used for quantitative detection of genomic deletions, duplications and point mutations. Multiplex ligation-dependent probe amplification discriminates sequences that differ even by a single nucleotide and can be used to detect known mutations. Methods for use in multiplex ligation-dependent amplification are described in Yau S C, et al., Accurate diagnosis of carriers of deletions and duplications in Duchenne/Becker muscular dystrophy by fluorescent dosage analysis, J Med Genet. 33(7):550-558 (1996); Procter M, et al., Molecular diagnosis of Prader-Willi and Angelman syndromes by methylation-specific melting analysis and methylation-specific multiplex ligation-dependent probe amplification, Clin Chem 52(7):1276-1283 (2006); Bunyan D J, et al., Dosage analysis of cancer predisposition genes by multiplex ligation-dependent probe amplification, Br J Cancer 91(6):1155-1159 (2004); U.S. Pub. 2012/0059594; U.S. Pub. 2009/0203014; U.S. Pub. 2007/0161013; U.S. Pub. 2007/0092883; and U.S. Pub. 2006/0078894, the contents of which are hereby incorporated by reference in their entirety.

Methods for detecting genetic markers at a site known to be associated with a genetic condition are useful in conjunction with the invention. Genetic markers can be detected using various tagged oligonucleotide hybridization technologies using, for example, microarrays or other chip-based or bead-based arrays. In some embodiments, a sample from an individual is tested simultaneously for multiple (e.g., thousands) genetic markers. Microarray analysis allows for the detection of abnormalities at a high level of resolution. An array such as an SNP array allows for increased resolution to detect copy number changes while also allowing for copy neutral detection (for both uniparental disomy and consanguinity). Detecting variants through arrays or marker hybridization is discussed, for example, in Schwartz, S., Clinical utility of single nucleotide polymorphism arrays, Clin Lab Med 31(4):581-94 (2011); Li, et al., Single nucleotide polymorphism genotyping and point mutation detected by ligation on microarrays, J Nanosci Nanotechnol 11(2):994-1003 (2011). Reverse dot blot arrays can be used to detect autosomal recessive disorders such as thalassemia and provide for genotyping of wild-type and thalassemia DNA using chips on which allele-specific oligonucleotide probes are immobilized on membrane (e.g., nylon). Assay pipelines can include array-based tests such as those described in Lin, et al., Development and evaluation of a reverse dot blog assay for the simultaneous detection of common alpha and beta thalassemia in Chinese, Blood Cells Mol Dis 48(2):86-90 (2012); Jaijo, et al., Microarray-based mutation analysis of 183 Spanish families with Usher syndrome, Invest Ophthalmol Vis Sci 51(3):1311-7 (2010); and Oliphant A. et al., BeadArray technology: enabling an accurate, cost-effective approach to high-throughput genotyping, Biotechniques Supp1:56-8, 60-1 (2002). DNA arrays in genetic diagnostics are discussed further in Yoo, et al., Applications of DNA microarray in disease diagnostics, J Microbiol Biotechnol 19(7):635-46 (2009); U.S. Pat. No. 6,913,879; U.S. Pub. 2012/0179384; and U.S. Pub. 2010/0248984, the contents of which are hereby incorporated by reference in their entirety.

In other embodiments, a variant (e.g., an SNP or indel) can be identified using oligonucleotide ligation assay in which two probes are hybridized over an SNP and are ligated only if identical to the target DNA, one of which has a 3′ end specific to the target allele. The probes are only hybridized in the presence of the target. Product is detected by gel electrophoresis, MALDI-TOF mass spectrometry, or by capillary electrophoresis. This assay has been used to report 11 unique cystic fibrosis alleles. Schwartz, et al., Identification of cystic fibrosis variants by polymerase chain reaction/oligonucleotide ligation assay, J Mol Diag 11(3):211-215 (2009). Oligonucleotide ligation assay for use in pipelines is described further in U.S. Pub. 2008/0076118 and U.S. Pub. 2002/0182609, the contents of which are hereby incorporated by reference in their entirety.

In some embodiments, results of the genetic sequence are provided according to a systematic nomenclature. For example, a variant can be described by a systematic comparison to a specified reference (i.e., a reference allele) which is assumed to be unchanging and identified by a unique label such as a name or accession number. For a given gene, coding region, or open reading frame, the A of the ATG start codon is denoted nucleotide +1 and the nucleotide 5′ to +1 is −1 (there is no zero). A lowercase g, c, or m prefix, set off by a period, indicates genomic DNA, cDNA, or mitochondrial DNA, respectively.

A systematic name can be used to describe a number of variant types including, for example, substitutions, deletions, insertions, and variable copy numbers. A substitution name starts with a number followed by a “from to” markup. Thus, 199A>G shows that at position 199 of the reference sequence, A is replaced by a G. A deletion is shown by “del” after the number. Thus 223delT shows the deletion of T at nt 223 and 997-999del shows the deletion of three nucleotides (alternatively, this mutation can be denoted as 997-999delTTC). In short tandem repeats, the 3′ nt is arbitrarily assigned; e.g. a TG deletion is designated 1997-1998delTG or 1997-1998del (where 1997 is the first T before C). Insertions are shown by ins after an interval. Thus 200-201insT denotes that T was inserted between nts 200 and 201. Variable short repeats appear as 997(GT)N-N′. Here, 997 is the first nucleotide of the dinucleotide GT, which is repeated N to N′ times in the population.

Variants in introns can use the intron number with a positive number indicating a distance from the G of the invariant donor GU or a negative number indicating a distance from an invariant G of the acceptor site AG. Thus, IVS3+1C>T shows a C to T substitution at nt +1 of intron 3. In any case, cDNA nucleotide numbering may be used to show the location of the mutation, for example, in an intron. Thus, c.1999+1C>T denotes the C to T substitution at nt +1 after nucleotide 1997 of the cDNA. Similarly, c.1997-2A>C shows the A to C substitution at nt−2 upstream of nucleotide 1997 of the cDNA. When the full length genomic sequence is known, the mutation can also be designated by the nt number of the reference sequence.

The above description of techniques and instruments for performing genetic analysis should be seen as exemplary. The methods and systems of the invention can operate independently of specific techniques for acquiring the genetic information.

### EXAMPLE

### Example 1

### Identifying Contamination in a Genetic Sample

As an example of the methods of the invention, a set of sequences known to be free from contamination was used to build a null distribution of allelic fractions for polymorphic loci. A sample that was known to be contaminated with foreign alleles was then scored in comparison to the known distribution.

A null set was used to determine allelic fraction distributions for 39 known polymorphic loci. The null set was based on sequences from 60 previous production runs, each run containing 10 to 75 unique samples. The large quantity of data allowed allelic fractions to be determined for homozygous and heterozygous genotypes at the 39 polymorphic loci. After the null set distribution was established for each genotype, the allelic fractions for each production run sample were individually compared to the null distribution for the identified genotype (see, e.g., steps 130-180 of FIG. 1). For each sample a z-score was calculated for each loci of the sample, and a summary score (mean z-score) was calculated using the z-scores all of the loci for each production run sample.

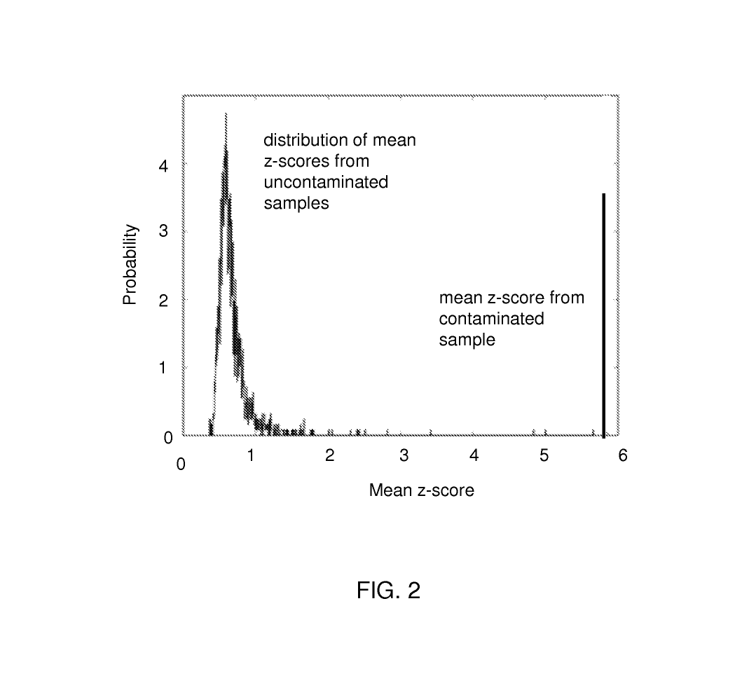

The distribution of mean z-scores for the production run samples can be seen as a large peak at approximately 0.75 in FIG. 2. Overall, the distribution of sample summary scores is clustered narrowly, having a full-width at half maximum of approximately 0.4. However a few outliers (e.g., small peaks between 3 and 6) indicate that some production samples may have sampling errors or other errors.

To test the methods of the invention, a sequence from a sample known to have been contaminated by foreign genetic material was scored against the null distribution. Again, following the steps outlined in FIG. 1, loci were located in the sample, and the relevant allelic fractions were scored against the null distribution of allelic fractions for each locus. The collected z-scores were then averaged to establish a mean z-score, which was 5.85, shown as the bold line on the right-hand side of the graph in FIG. 2. Clearly, the contaminated sample stands out from the samples of the null set. A p-value calculated from the data shown in FIG. 2, was less than 0.001, further evidence that the sample was contaminated.

Thus, the example illustrates that the methods of the invention can be used to successfully distinguish a sample that has been contaminated by foreign genetic material.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A method for identifying contamination in a sample, comprising:
obtaining a sample comprising genomic material;
determining a sample allelic frequency at one or more polymorphic loci in the genomic material;
comparing said sample allelic frequency to a reference allelic frequency expected to be present in said sample; and
identifying genomic contamination in said sample if there is a statistically-significant difference between said sample allelic frequency and said reference allelic frequency.

2. The method of claim 1, wherein said genomic material is selected from DNA and RNA.

3. The method of claim 1, wherein said sample allelic frequency is determined across a plurality of polymporphic loci and said comparing step comprises comparing the sample allelic frequency to a reference allelic frequency across said plurality of polymorphic loci that would be expected in the absence of contamination.

4. The method of claim 1, wherein said sample allelic frequency is determined by sequencing nucleic acid comprising said polymorphic loci.

5. The method of claim 4, wherein said sequencing comprises next-generation sequencing.

6. The method of claim 1, wherein said determining step comprises genotyping.

7. The method of claim 1, wherein said determining step comprises comparing ratios of fluorescence intensities.

8. The method of claim 7, further comprising contacting said genomic material with fluorescently-labeled hybridization probes.

9. The method of claim 1, wherein said comparing step comprises creating a summary statistic based upon said sample allelic frequency and said reference allelic frequency.

10. The method of claim 9, wherein the summary statistic is selected from mean z-score, median z-score, and maximum z-score.

11. The method of claim 1, wherein said reference allelic frequency expected to be present in said sample is based upon a collection of sequence data known to be substantially free from contamination.

12. The method of claim 1, wherein said reference allelic frequency expected to be present in said sample is based upon a biomarker observed in an organism from which the sample originated.

13. The method of claim 1, wherein the genomic material is from a mammal.

14. The method of claim 13, wherein the genomic material is from a human.
